(19) United States
(12) Patent Application Publication
(10) Pub. No.: US 2009/0149724 A1
(43) Pub. Date: Jun. 11, 2009

(54) SYSTEM AND METHOD FOR PREDICTING SEPTIC SHOCK

(75) Inventors: Roger G. Mark, Cambridge, MA (US); Dewang V. Shavdia, Reistertown, MD (US)

Correspondence Address: WOLF GREENFIELD & SACKS, P.C., 600 ATLANTIC AVENUE, BOSTON, MA, 02210-2206, US

(73) Assignee: Massachusetts Institute of Technology, Cambridge, MA (US)

(21) Appl. No.: 12/329,156

(22) Filed: Dec. 5, 2008

Related U.S. Application Data
(60) Provisional application No. 61/005,477, filed on Dec. 5, 2007.

(51) Int. Cl. A61B 5/00; G06F 17/10; G06G 7/60

(52) U.S. Cl. 600/301; 703/2

(57) ABSTRACT

A model uses variables that are normally collected from a subject during treatment to determine whether the subject may be progressing toward septic shock.

### RELATED APPLICATIONS

This application claims priority to U.S. Provisional Application Ser. No. 61/005,477 filed Dec. 5, 2007.

### BACKGROUND

Sepsis is a leading cause of death in healthcare facilities with mortality rates upward of 50% for some intensive care units. Sepsis occurs as a result of an infection, such as a bacterial, fungal, viral, or parasitic infection. At some point, the infection causes a progression to Systemic Inflammatory Response Syndrome, or SIRS. SIRS has been identified by some as an inflammatory response to the infection that uncontrollably cascades, causing damage to bystanding tissue that may ultimately results in organ dysfunction.

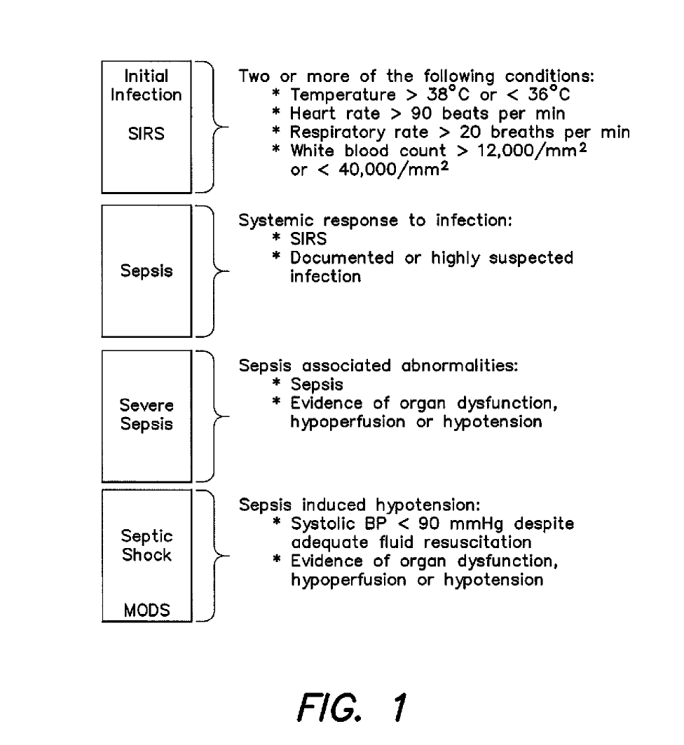

The stages associated with sepsis are represented in FIG. 1. At the beginning of the progression, the initial infection results in SIRS. SIRS, as defined by a 1991 ACCP/SCCM Consensus Conference, occurs when a subject has two or more clinical abnormalities of the group that includes: a temperature greater than 38 degrees Celsius or lower than 36 degrees Celsius; a heart rate greater than 90 beats per minute; a respiratory rate greater than 20 breathes per minute; and a white blood cell count greater than 12,000 cells per cubic millimeter or less than 4,000 cells per cubic millimeter. SIRS progresses to sepsis when a subject has two or more of the above discussed clinical abnormalities, and the response can be traced to a documented or highly suspect source of infection. When there is also evidence of organ dysfunction or hypoperfusion/hypotension, such as lactic acidosis, or an acute change in mental status, the subject is deemed to be experiencing severe sepsis. The condition is deemed to have progressed to septic shock when the subject exhibits arterial hypotension (i.e., a systolic blood pressure less than 90 mmHg or a 40 mmHg drop from baseline systolic blood pressure) despite fluid resuscitation.

Effective treatments for septic shock are in existence and are available. Such treatments may include the administration of antibiotics and vasopressors/inotropes, surgical drainage of infected fluids, and fluid replacement. Treatment may also be directed to various dysfunctional organs, like hemodialysis for kidneys, mechanical ventilation for lungs, and/or blood transfusions and drug and fluid therapy for circulatory failures.

### SUMMARY

The applicant has appreciated that the high mortality rates associated with sepsis, and particularly for septic shock, may not be related to the lack of an effective treatment, but rather to difficulty and/or delays in predicting and/or identifying the onset of septic shock such that treatments may be administered.

Aspects of the present invention relate to a model that uses variables that are normally collected from a subject during treatment. The model uses the variables to determine whether the subject may be progressing toward septic shock.

According to one aspect, a method of identifying when a subject is at an elevated risk for septic shock is disclosed. The method includes measuring a plurality of time varying physiological variables of the subject and providing the plurality of time varying physiological variables to a model. The method also includes calculating, with the model, an output indicator based on the plurality of time varying physiological variables received by the model and identifying that the subject is at an elevated risk for septic shock when the output indicator, provided by the model, exceeds a threshold value.

According to another aspect, a system for identifying when a subject is at an elevated risk for septic shock includes an input device for receiving a plurality of time varying physiological variables of the subject. The system also includes a controller that calculates, based on the plurality of time varying physiological variables and a model, an output indicator. An output device provides an alert when the output indicator exceeds a threshold value indicating that the subject is at an elevated risk for septic shock.

According to yet another aspect, a method of constructing a model to identify when a subject is at an elevated risk for septic shock includes gathering measurements of a group of time varying physiological variables at different times from subjects that experienced at least one of SIRS, sepsis, severe sepsis, or septic shock. Times are identified at which the subjects exhibited an indicator of septic shock. Combinations of the time varying physiological variables chosen and constants associated with the time varying physiological variables are defined that when multiplied by corresponding time varying physiological variables and summed, produce an output indicator that represents when a corresponding subject was at an elevated risk for septic shock.

### DETAILED DESCRIPTION

The accompanying drawings are not intended to be drawn to scale. In the drawings, each identical or nearly identical component that is illustrated in various figures may be represented by a like numeral. For purposes of clarity, not every component may be labeled in every drawing. In the drawings:

FIG. 1 shows the stages associated with sepsis.

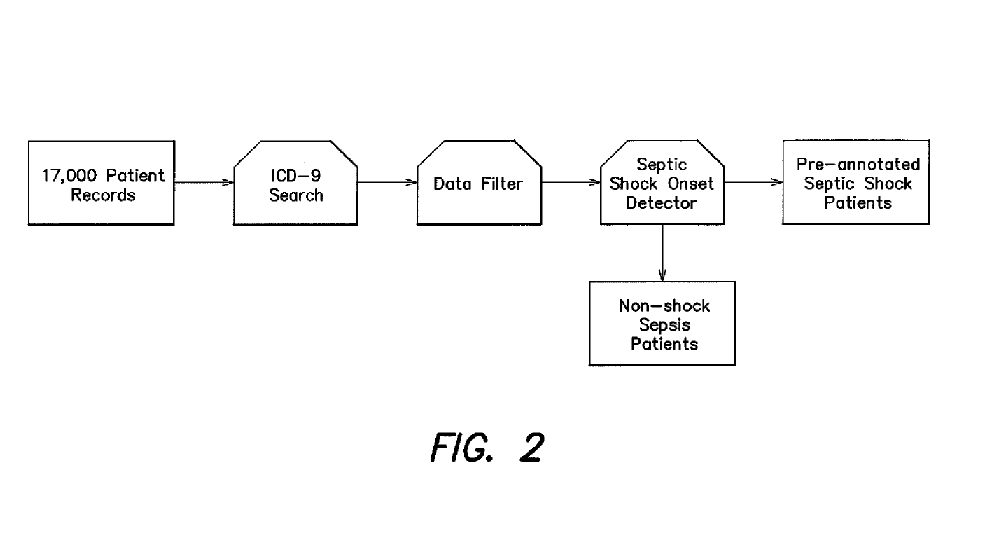

FIG. 2 shows a flow diagram of dataset being annotated through an automated process, prior to being used to produce a model, according to one embodiment.

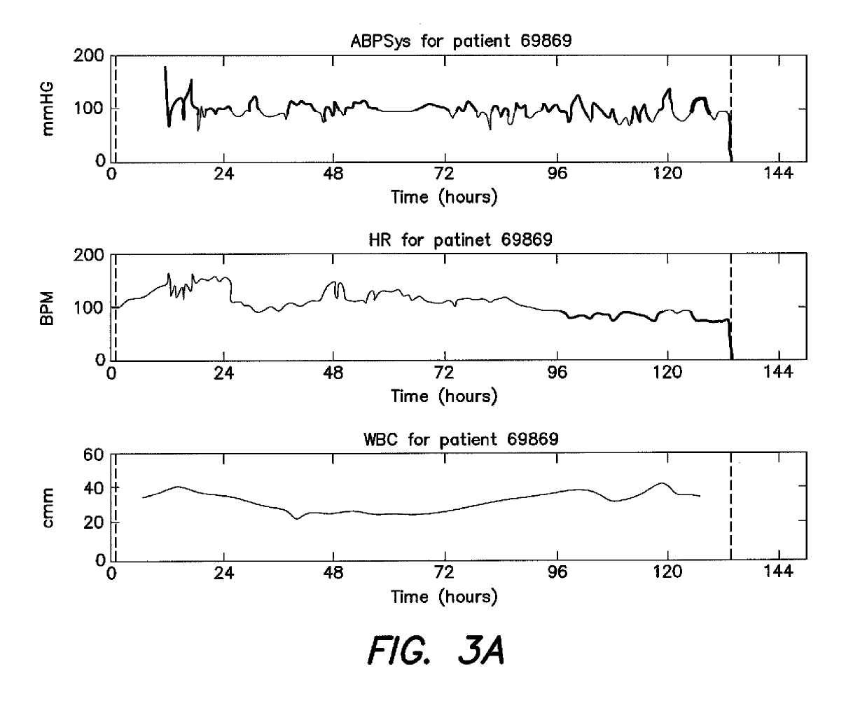

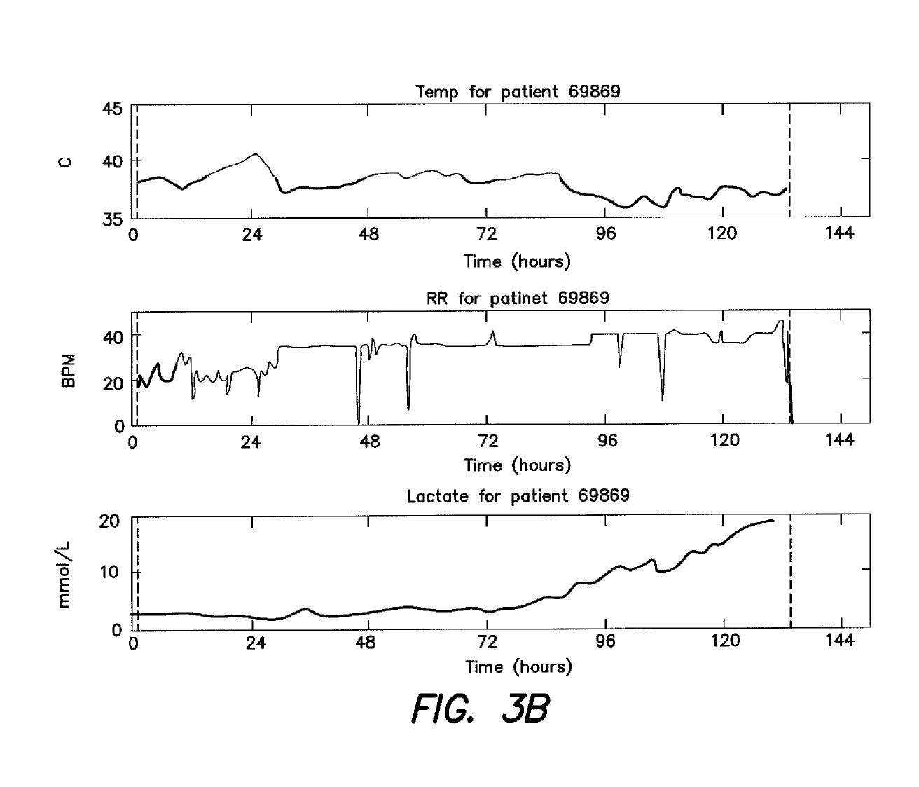

FIGS. 3a and 3b show data for one subject that was included in the dataset of the embodiment of FIG. 2, and who experienced septic shock.

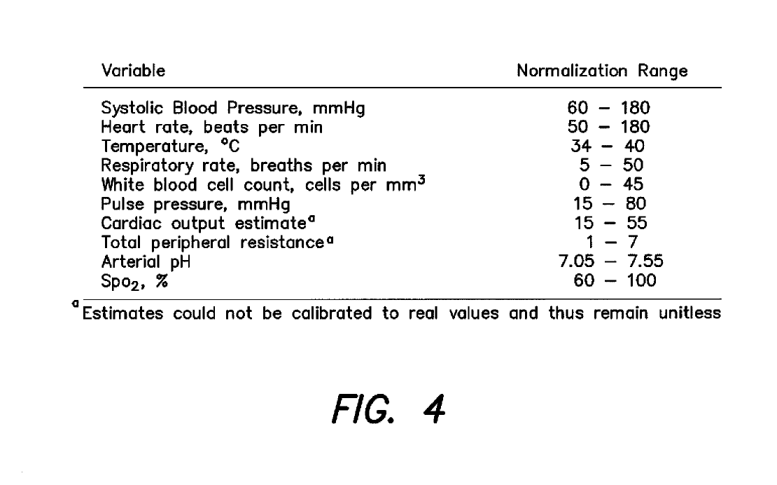

FIG. 4 shows ranges that were used for data normalization in a dataset, according to one embodiment.

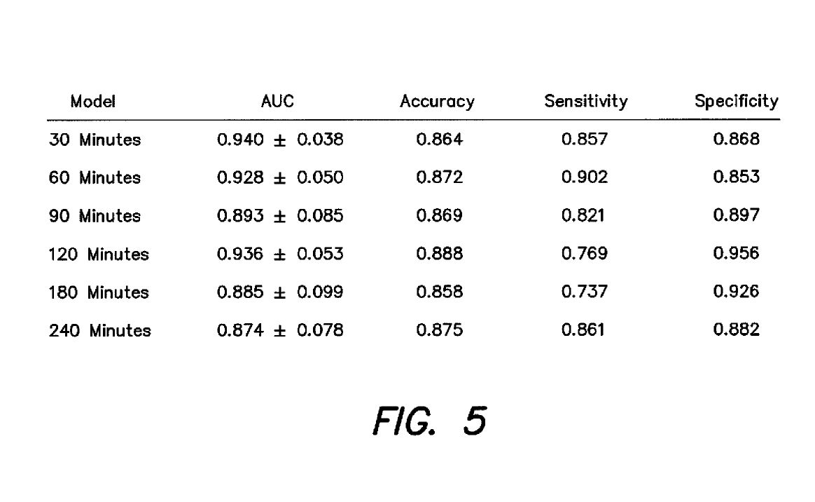

FIG. 5 shows the accuracy, sensitivity, and specificity of a few models, according to some embodiments.

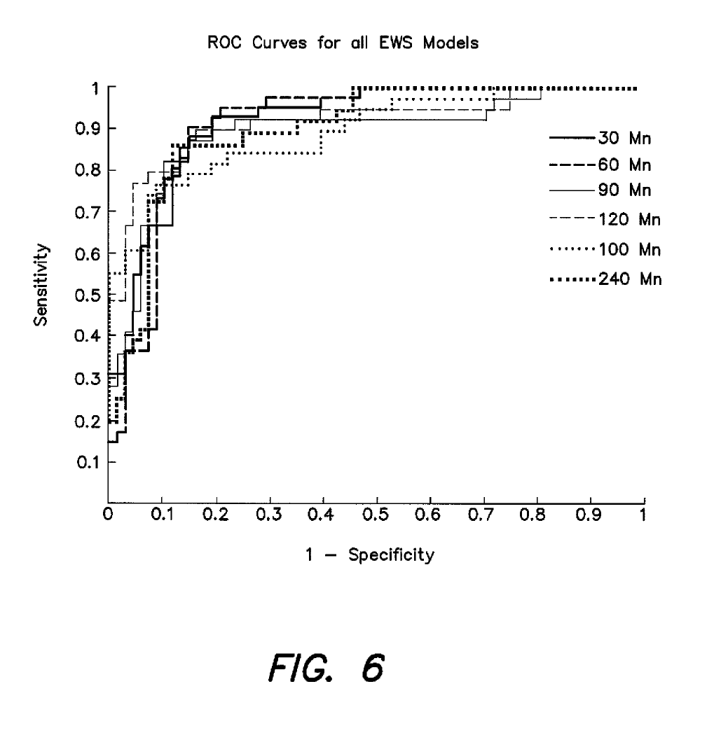

FIG. 6 shows plots of receiver operating curves for each of the models represented in FIG. 5.

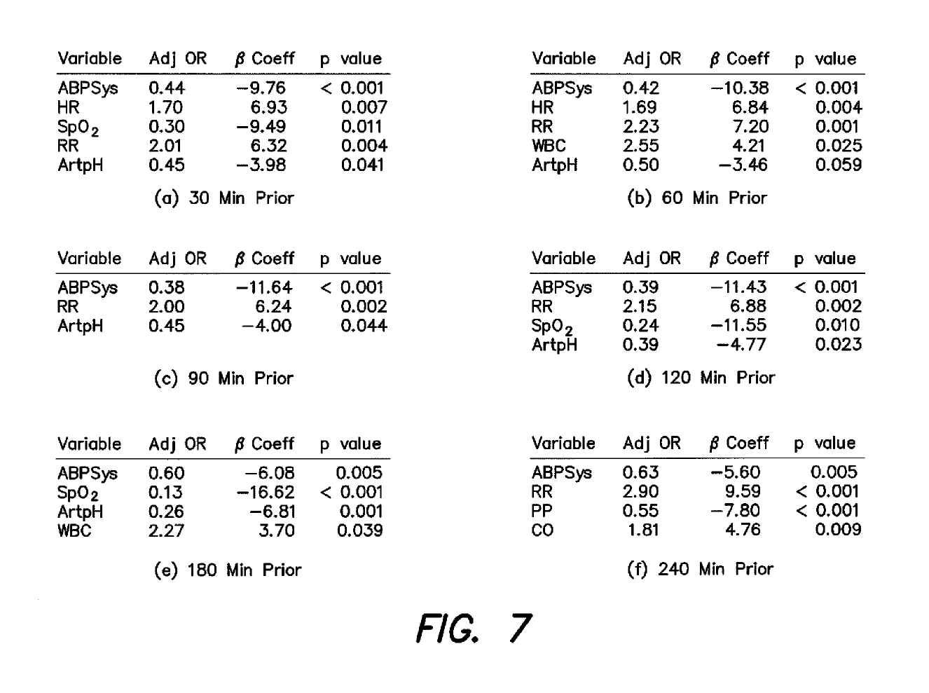

FIG. 7 shows the k-best variables selected by models represented in FIG. 6.

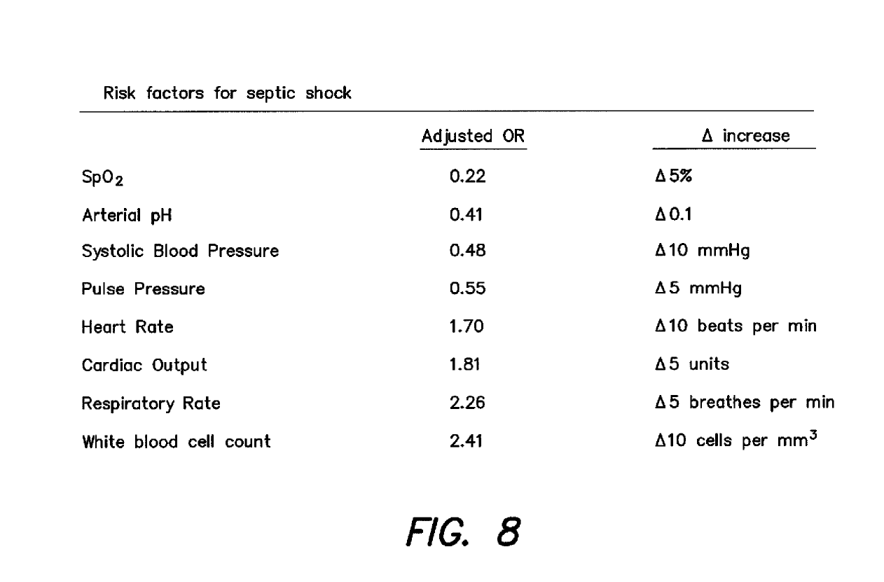

FIG. 8 shows the adjusted odds ratios associated with different variables associated with models, according to some embodiments.

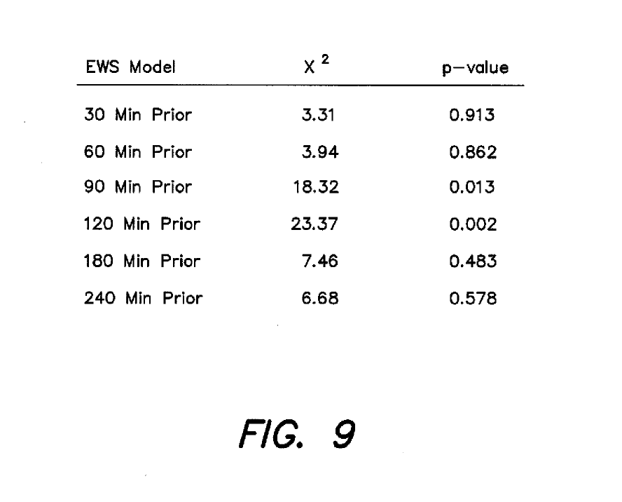

FIG. 9 shows the results from a Hosmer-Lemeshow goodness-of-fit testing for two models.

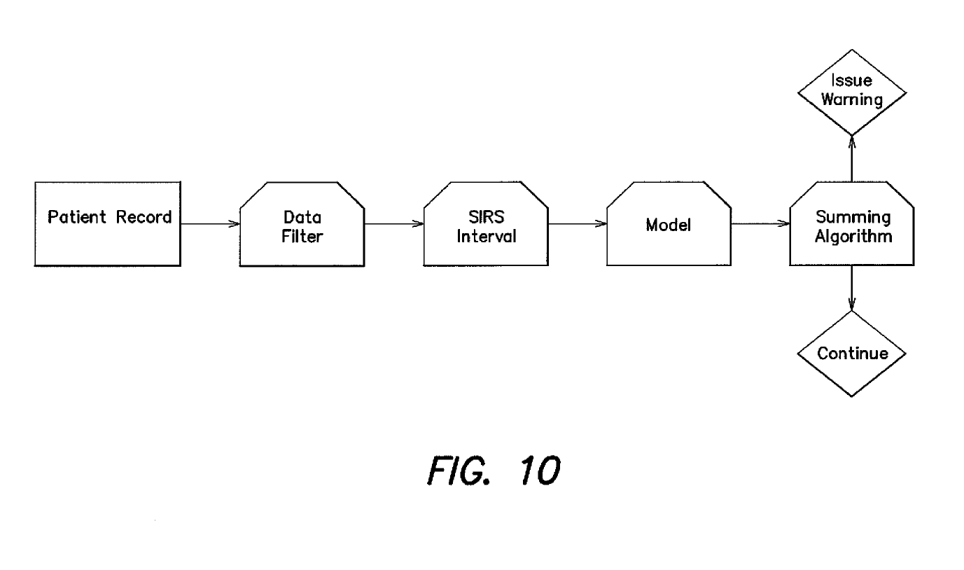

FIG. 10 shows a flow diagram of testing performed on models, according to one embodiment.

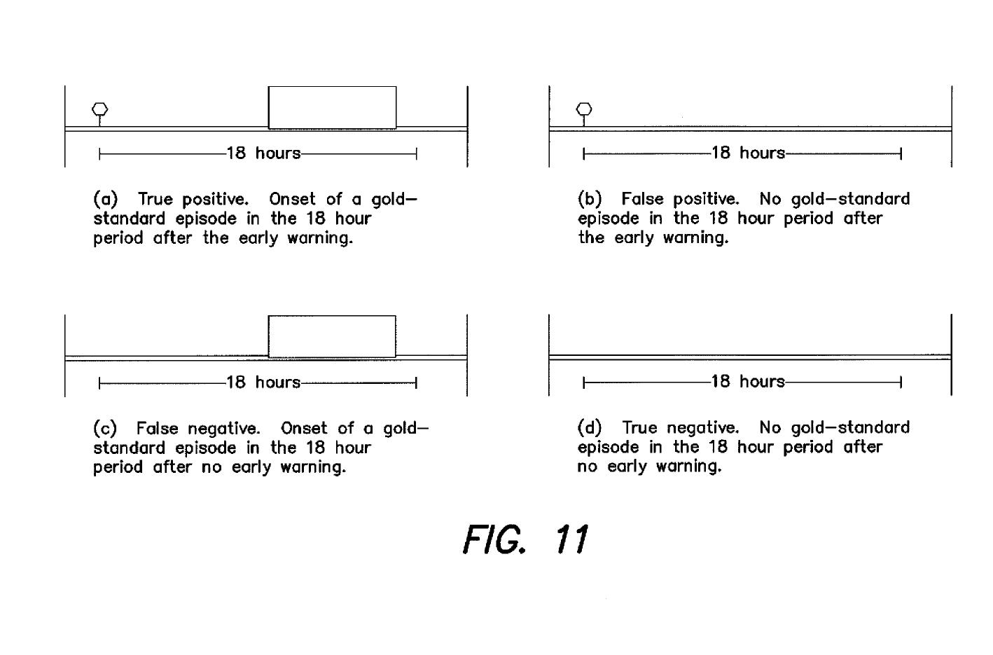

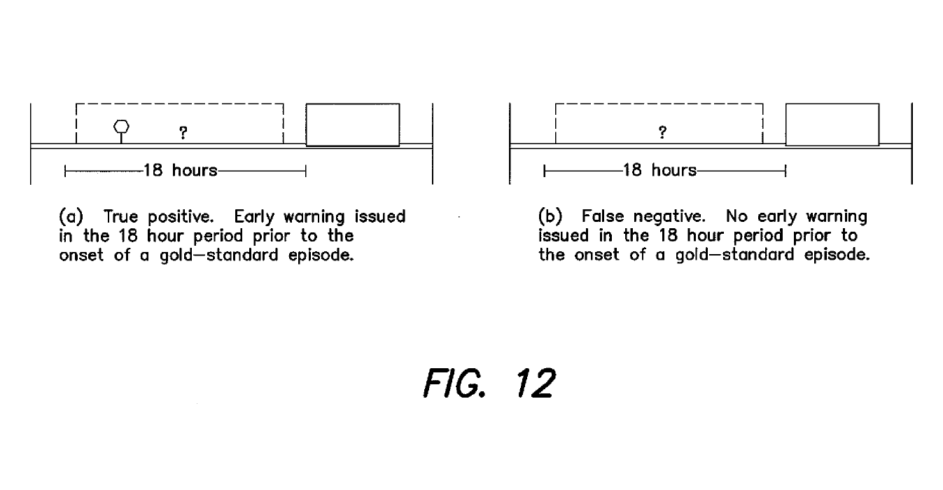

FIG. 11 shows a graphical depiction of definitions of true positive, false positive, true negative, and false negative occurrences used to describe model performance, according to some embodiments.

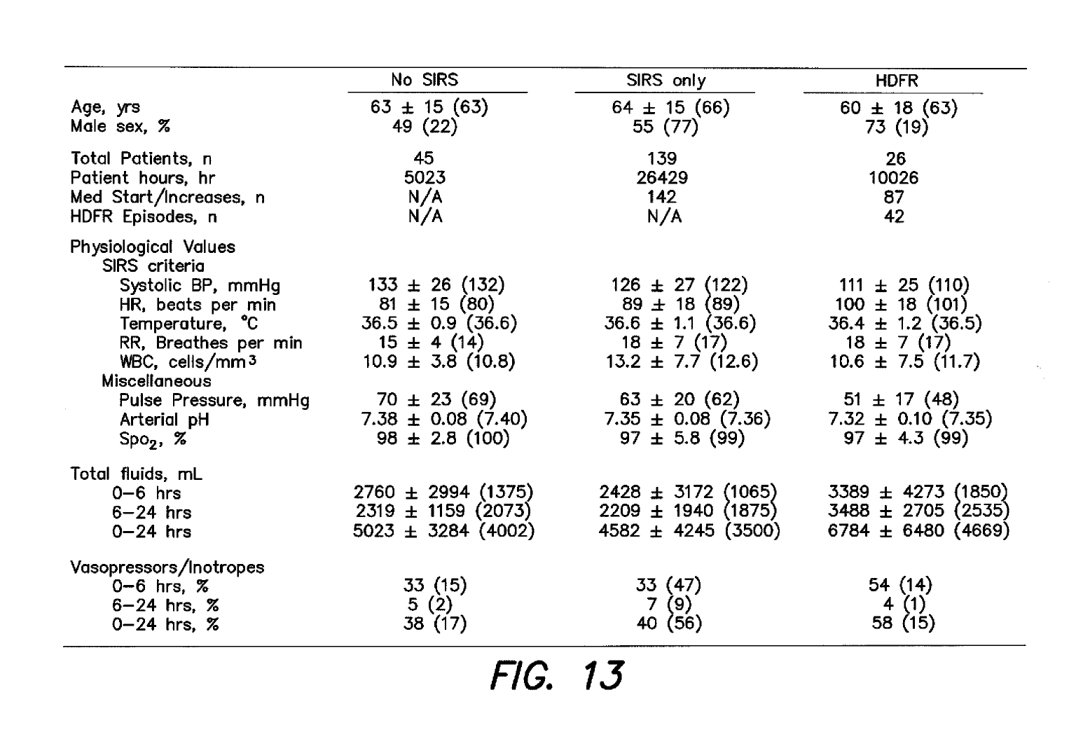

FIG. 12 shows a graphical depiction of definitions of true positive and false negative occurrences used to describe model performance, according to some embodiments.

FIG. 13 shows the results of testing performed on models, according to one embodiment.

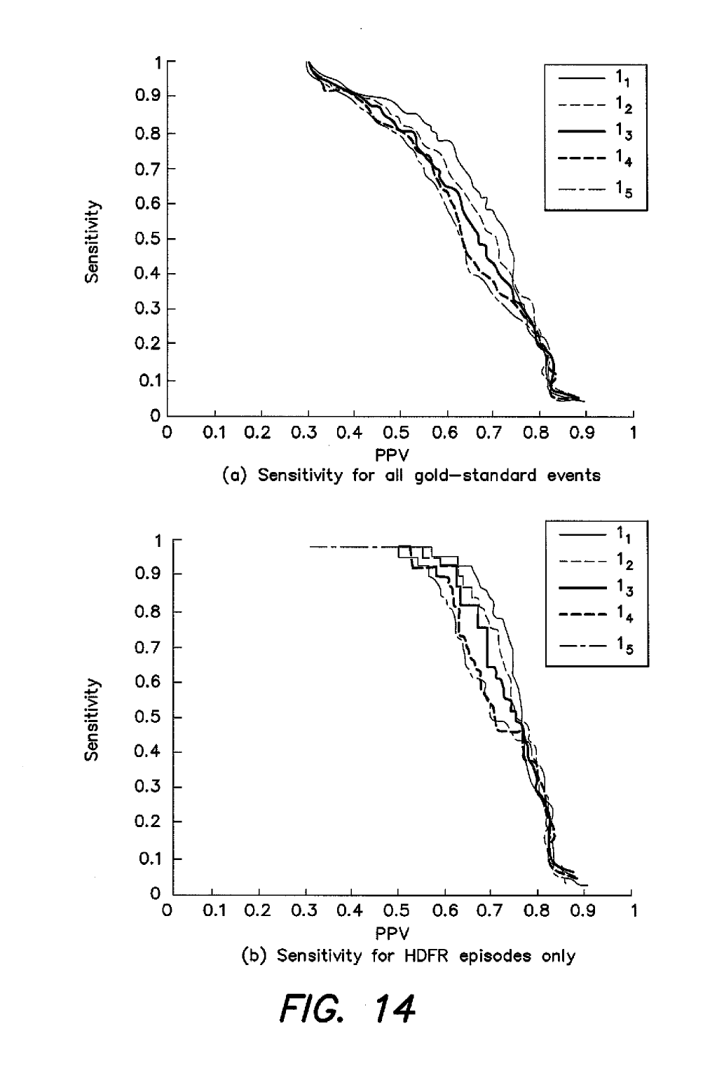

FIG. 14 shows a plot for sample sensitivity versus PPV and a plot of sensitivity for hypotension despite fluid resuscitation (HDFR) episodes versus PPV for a model, according to one embodiment.

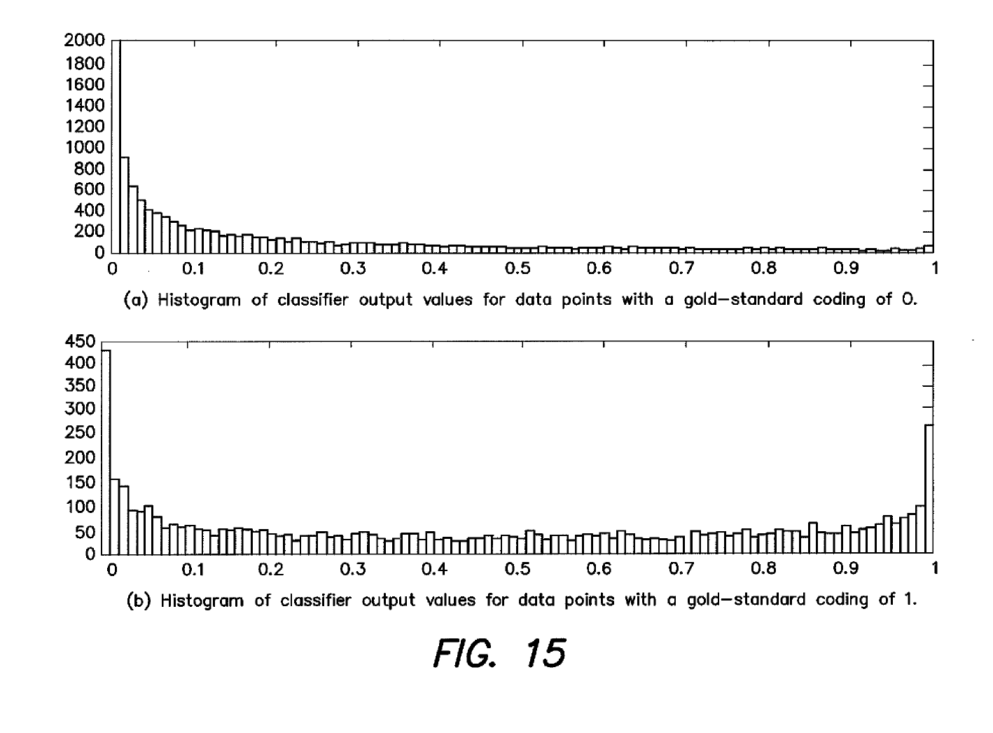

FIG. 15 includes histograms of classifier output values for data points with a gold-standard coding of 0 and 1, respectively.

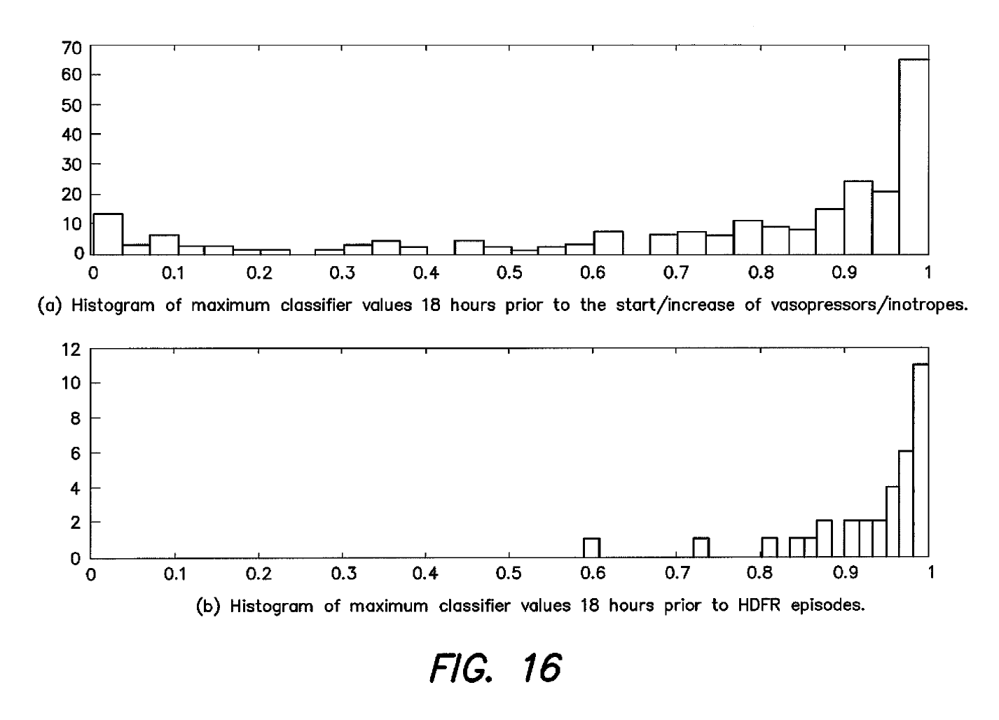

FIG. 16 includes histograms of maximum classifier output values in 18 hours prior to gold-standard episodes, for various models.

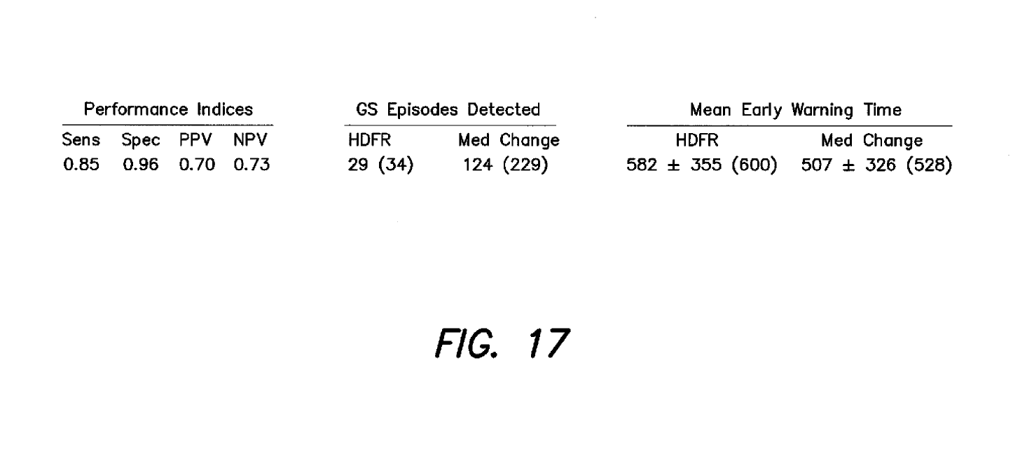

FIG. 17 shows performance indices for some model embodiments, when run with an output indicator threshold of 0.87 (on a scale between 0 and 1).

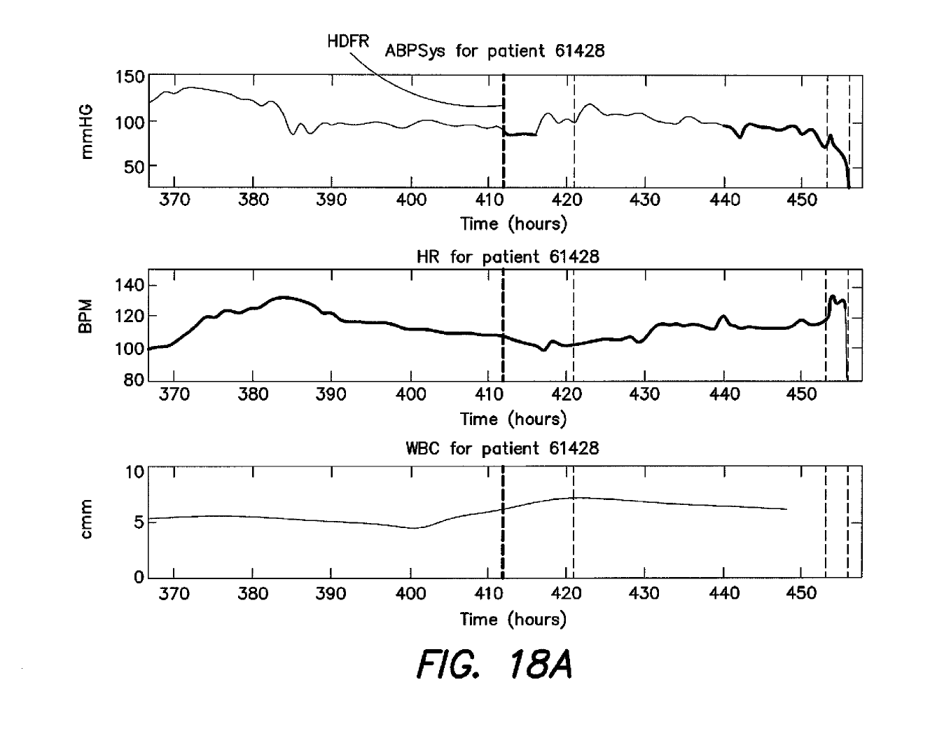

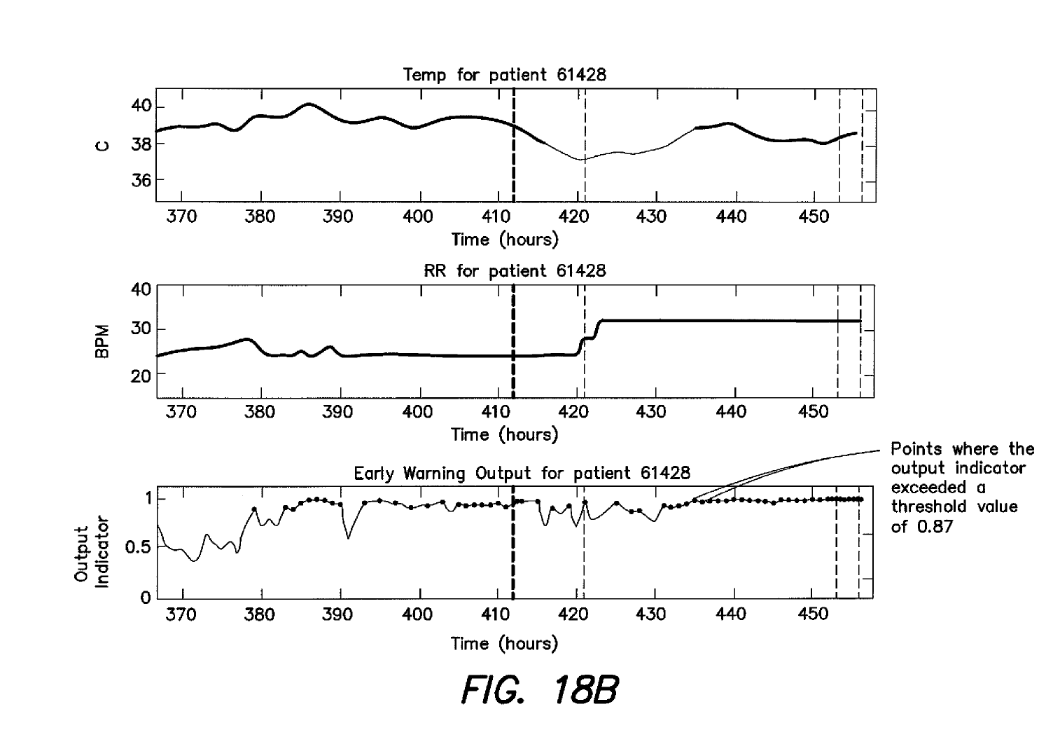

FIGS. 18a and 18b shows a sample patient run tracking systolic blood pressure, heart rate, white blood cell count, temperature, and respiratory rate.

### DETAILED DESCRIPTION

According to some aspects of the invention, time varying physiologic data is collected from a subject and is used to predict whether and/or when the subject may be progressing toward septic shock. The data may include time varying physiologic information that is normally collected during treatment of the subject. The physiologic information may be used to calculate an output indicator, that when in excess of a threshold value, predicts that the subject has a high likelihood of experiencing hypotension despite fluid resuscitation while exhibiting SIRS: the hallmark of the onset of septic shock.

Aspects of the invention also relate to a method of constructing a model to predict whether and/or when the subject may experience hypotension despite fluid resuscitation while exhibiting SIRS. The model may be constructed using time varying physiological data from prior subjects that experienced various phases of sepsis, including SIRS, sepsis, severe sepsis, and/or septic shock. Relationships may be identified between combinations of the variables and whether and when subjects have exhibited indicators of septic shock, such as hypotension despite fluid resuscitation and/or the administration of vasopressors/inotropes. According to some embodiments, the model may be based on multivariate logistic regression approach, although other approaches may also be used.

Aspects of the invention may be embodied in a system that may be used to collect time varying physiologic data from a subject. The data may used by a model that is incorporated into the system and that predicts whether and/or when the subject may be progressing toward septic shock. The system may include an input device that receives the time varying physiological data from the subject and a controller that calculates, based on a model, whether the subject is at an elevated risk for septic shock. The system may also include an output indicator that may be used to alert a healthcare professional when the subject is at an elevated risk for septic shock.

Methods and systems of the present invention may utilize information that is normally collected during treatment of a subject. Some non-limiting examples of such information include blood pressure (systolic and/or diastolic), heart rate, temperature, respiratory rate, arterial pH, oxygen saturation of arterial blood (Sp02), and/or white blood cell count, to name a few. Additionally or alternately, information may be calculated from this information, such as an estimated cardiac output and/or estimated total peripheral resistance, and then provided to a model. The model may use this information to provide an output indicator, such as one or more numbers, that when exceeded alert a healthcare professional that the subject is likely to progress toward septic shock.

Embodiments of the invention may be suited to utilize data that is typically gathered from a subject that is under treatment in an intensive care unit In this respect, various embodiments may be instituted without increasing the workload for medical professionals that are treating a subject. As discussed in greater detail below, mechanisms that are already in place within a typical intensive care unit or other treatment center may be used to provide information to embodiments of the invention.

Data collected through sensors that are normally placed on the subject may be passed automatically to an input device of the system. By way of example, blood pressure readings from an arterial catheter that are recorded at regular intervals may be provided to the input device of the system. Other variables that are taken from a subject automatically, like blood pressure, may include temperature, pulse oximetry oxygen saturation, cardiac output, and the like. It is to be appreciated, however, that such data gathering need not occur automatically, and that healthcare professionals may instead collect this information manually, and enter the data into an input device of the system. Alternately, healthcare professionals may collect some or all of the data manually, and perform manual calculations based on the model, as aspects of the invention are not limited in this regard.

Time varying physiological data may also be derived from lab testing. Labs that are typically taken on a periodic basis during a subject's stay in a treatment center may prove particularly conducive for use by the system. Some of such variables include, but are not limited to, arterial pH and white blood cell counts. The input device for various embodiments of the system may include a data input feature, such as a keypad, where such lab values may be entered by a healthcare professional. Additionally or alternately, values for these variables may be conveyed electronically to an input device of the system, or may be used in manual calculations, as aspects of the invention are not limited in this respect.

Data that is collected directly from the patient may also be used to provide estimates for some time varying physiological data that is, in turn, used by the system. By way of example, cardiac output may be calculated, such as by using the Liljestrand technique, and then provided to the model for use in predicting whether the subject will experience septic shock. Total peripheral resistance is yet another example of a time varying physiological variable that may be calculated, rather than measured, and provided to a model for use in predicting septic shock.

Models for predicting the whether a subject is likely to experience septic shock may be constructed in various manners. According to some embodiments, the model may be a multivariate logistic regression model, constructed in the form of Eq. 1.

$$Output = \frac{{e}^{( {β}_{0} + {β}_{1} {x}_{1} + {β}_{2} {x}_{2} + … )}}{( 1 + {e}^{( {β}_{0} + {β}_{1} {x}_{1} + {β}_{2} {x}_{2} + … )} )} \qquad Eq . 1$$

Where:
Output: Output indicator
x: Value of a time varying variable associated with the corresponding subscript
β: Constant associated with corresponding time varying variable

Multivariate logistic regression models, like those associated with Eq. 1, may prove beneficial in that they may more readily be created using machine learning approaches. According to some embodiments of the invention, datasets of subjects who experienced septic shock may be created and then used with machine learning approaches to identify appropriate β (Beta) values for various combinations of variables. Some exemplary approaches are discussed below under the Examples section.

It is to be appreciated that while models may be constructed that utilize multivariate logistic regression, like that associated with Eq. 1, aspects of the invention are not limited in this respect. Models constructed according to other techniques, such as linear regression or neural networks, among others, may also be utilized. Moreover, although Eq. 1 represents a model that uses more than two time varying physiological values, other models may use fewer variables. There are no bounds on the number of variables that may be used, such that as few as a single variable, and as much as 3 or more, 5 or more, 7 or more, 10 or more, or even 20 or more variables may be utilized, according to some embodiments.

Data may be processed prior to being used in any calculations by a model. According to some embodiments, the time varying physiological values are normalized to a scale between 0 and 1 prior to being provided the model. In such embodiments, a range of expected values for a typical variable may be determined ahead of time. Measured values that lie within this range may be scaled to an appropriate point between 0 and 1 (or another appropriate range of values). Data points that lie below or above the expected range may be set to 0 or 1, respectively, or discarded from the calculation altogether. In this respect, such embodiments may filter data, somewhat, to prevent outliers from affecting calculations too drastically. It is to be appreciated that other data filtering techniques may also be used, as aspects of the invention are not limited in this manner.

A threshold value may be identified such that healthcare professionals may be alerted to the predicted onset of septic shock when the output of the model exceeds the threshold. The threshold may be identified empirically, using databases of information on prior subjects that experienced septic shock and/or subjects that experienced various stages of sepsis without progressing to septic shock. Historical information as to when such subjects experienced hypotension despite fluid resuscitation or when vasopressor and/or inotropes were administered to the subjects may prove useful in defining the point at which such subjects transitioned from sepsis or severe sepsis to septic shock.

Various embodiments may provide updated output according to different rates. According to some embodiments, an output indicator is calculated at a rate consistent with the variable that is sampled most frequently. In such approaches, the latest valid value may be used for variables that are sampled less frequently. Typical variables that are sampled more frequently include those that are measured automatically, like heart rate, blood pressure, temperature, and pulse oximetry oxygen saturation, to name a few. Variables that are typically sampled on longer time frames may include white blood cell count and other variables that are determined by lab test.

According to other embodiments, the output indicator may be recalculated at rates that are less frequent than those associated with the variable that is sampled most frequently. Some approaches may also include recalculating the output indicator according to a schedule that is not tied to the sampling rates of any variables that are provided to the model, such as by recalculating the output indicator every hour with the latest values that are available.

Each calculation of the output indicator may be an independent event, or may incorporate results from prior calculations, or prior values of variables used in those calculations. According to some approaches, the time varying physiological variables may be combined as a weighted sum, as suggested by Eq. 2 below. Some examples of alternate approaches include exponentially weighted sums, linearly weighted sums, multiple consecutive values and unweighted addition, and multiple consecutive values and linearly weighted addition, as represented by Eq. 3-6 below, respectively. Equations 2 through 6 may be implemented by using any number of prior values (x). According to some embodiments, no prior values (n=1) and 4 prior values (n=5) are used.

$$\begin{matrix}Output = {\sum }_{i = 1}^{n} {x}_{i} & Eq . 2 \\ Output = {\sum }_{i = 1}^{n} {( {x}_{i} )}^{i} & Eq . 3 \\ Output = {\sum }_{i = 1}^{n} \frac{n - ( i - 1 )}{n} \cdot {x}_{i} & Eq . 4 \\ Output = {\sum }_{i = 1}^{n} {x}_{i - 1} \cdot {x}_{i} & Eq . 5 \\ Output = {\sum }_{i = 1}^{n} {x}_{i - 1} \cdot {x}_{i} + \frac{n - ( i - 1 )}{n - 1} \cdot {x}_{i - 1} & Eq . 6\end{matrix}$$

Various types of alarms may be activated once a threshold value is exceeded. In this respect, healthcare professionals may be alerted such that appropriate responses may be taken. Embodiments of the system may also direct the healthcare professional to the clinical abnormalities, as defined by SIRS or otherwise, that might help direct the caretaker in deciding upon an appropriate response.

According to some embodiments, a system includes a controller to make calculations, based on a model. The controller may receive information manually from a healthcare professional or automatically from any existing sensors and/or systems. The controller may control the system to calculate updated output indicators and to determine whether a threshold value has been exceeded. When the threshold value is exceeded, the controller may activate an alarm to alert a healthcare professional. The controller and model may be implemented in any of numerous ways. For example, in one embodiment the controller and model combination may be implemented using hardware, software or a combination thereof. When implemented in software, the software code can be executed on any suitable processor or collection of processors, whether provided in a single computer or distributed among multiple computers. It should be appreciated that any component or collection of components that perform the functions described herein can be generically considered as one or more controllers that control the functions discussed herein. The one or more controllers can be implemented in numerous ways, such as with dedicated hardware, or with general purpose hardware (e.g., one or more processors) that is programmed using microcode or software to perform the functions recited above. The one or more controllers may be included in one or more host computers, one or more storage systems, or any other type of computer that may include one or more storage devices coupled to the one or more controllers. In one embodiment, the controller includes a communication link to communicate wirelessly, or via electrical or optical cable, to a remote location. A human being may also act as a controller, making calculations by hand or with a calculator to determine an output indicator.

In this respect, it should be appreciated that one implementation of the embodiments of the present invention comprises at least one computer-readable medium (e.g., a computer memory, a floppy disk, a compact disk, a tape, etc.) encoded with one or more models in the form of a computer program (i.e., a plurality of instructions), which, when executed by the controller, performs the herein-discussed functions of the embodiments of the present invention. The computer-readable medium can be transportable such that the treatment protocol stored thereon can be loaded onto any computer system resource to implement the aspects of the present invention discussed herein. In addition, it should be appreciated that the reference to a model or controller which, when executed, performs the herein-discussed functions, is not limited to an application program running on a host computer.

The system may also comprise one or more sensors that receive information relating to time varying physiological variables from the subject and/or portions of the system itself. Such sensors may receive information regarding temperature, blood pressure, heart rate, respiratory rate, and the like.

Further, it should be appreciated that a computer may be embodied in any of a number of forms, such as a rack-mounted computer, a desktop computer, a laptop computer, or a tablet computer. Additionally, a computer may be embedded in a device not generally regarded as a computer but with suitable processing capabilities, including a Personal Digital Assistant (PDA), a smart phone or any other suitable portable or fixed electronic device.

Also, a computer may have one or more input and output devices. These devices can be used, among other things, to present a user interface. Examples of output devices that can be used to provide a user interface include printers or display screens for visual presentation of output and speakers or other sound generating devices for audible presentation of output Examples of input devices that can be used for a user interface include keyboards, and pointing devices, such as mice, touch pads, and digitizing tablets. As another example, a computer may receive input information through speech recognition or in other audible format.

Such computers may be interconnected by one or more networks in any suitable form, including as a local area network or a wide area network, such as an enterprise network or the Internet. Such networks may be based on any suitable technology and may operate according to any suitable protocol and may include wireless networks, wired networks or fiber optic networks.

Also, the various methods or processes outlined herein may be coded as software that is executable on one or more processors that employ any one of a variety of operating systems or platforms. Additionally, such software may be written using any of a number of suitable programming languages and/or conventional programming or scripting tools, and also may be compiled as executable machine language code or intermediate code that is executed on a framework or virtual machine.

The present invention is further illustrated by the following Examples, which in no way should be construed as further limiting.

### EXAMPLES

### Example 1

### Constructing a Dataset for Use in Building a Model to Predict Septic Shock

According to one example, a dataset was prepared for using an automated classification system to develop a model for predicting septic shock. The dataset was taken from a database that includes information from about 17,000 actual subjects during their stay in an intensive care unit. Data for subjects who had ICD-9 coding for septic shock were used to construct the dataset. A first set of subjects in the dataset experienced septic shock. For each subject of the first set, there was a documented time of transition from sepsis or severe sepsis to septic shock. Each of the subjects in the first set also had hypotension that was not secondary to another cause, like cardiogenic shock. A second set of subjects were included in the dataset for negative control. These subjects had severe sepsis, but did not transition to septic shock, and there was sufficient data to support that conclusion.

For each subject, four categories of clinical information were used in the dataset, including physiologic values, lab values, medications, and fluid administration. Only subjects that had nurse verified data were used in the dataset, meaning that a nurse entered or confirmed the value for the measurement of interest. Sampling rates for physiologic values, medications, and fluids varied from 15-120 minutes, with a mean and median of approximately 60 minutes. Sampling rates for lab values depended upon how often the measurements were requested by the clinician. In this example, nurse verified data was chosen to reduce the incidence of artifacts and other anomalies in the dataset. The sample rates above, for this example, may be frequent enough for a model that predicts the onset of septic shock, which may occur over a span of hours to days.

The physiologic values included for each subject in the dataset were systolic blood pressure, mean blood pressure, diastolic blood pressure, pulse pressure, central venous pressure, heart rate, temperature, respiratory rate, arterial pH, and pulse oximetry oxygen saturation. Cardiac output was calculated from physiologic values using the Liljestrand technique, and was included with the dataset. Total peripheral resistance was also calculated from the above physiologic values and included in the dataset.

Lab values included for each subject were lactate, white blood cell count, creatinine, creatine phosphokinase, and troponin.

Medication information in the dataset was limited to vasopressor/inotrope administration. This information was used in determining when a threshold value of the model output may be used to predict septic shock.

All fluid information for the subjects was included in the dataset, and was used to determine when the hypotension persisted for a particular subject, despite fluid resuscitation.

Prior to being used to produce a model, the dataset was annotated through an automated process, as represented in FIG. 2. The initial phase of the annotation process included building the dataset with the above described data for subjects that were coded under the International Classification of Diseases system (ICD-9) as having septic shock. The resulting data was then filtered to remove subjects that had inadequate data. For this particular example, adequate data was deemed to be at least 10 measured values of systolic blood pressure, heart rate, temperature, and respiratory rate. Additionally, subjects without at least two white blood cell counts were not included in the dataset. This data is associated with an amount of data that may be used to make a diagnosis of septic shock based on the 1991 ACCP/SCCM definition.

Each subject was then classified as either being a non-sepsis subject, a sepsis or severe sepsis subject that did not experience septic shock, or a septic shock subject. This classification was performed automatically, as represented by the block titled “septic shock onset detector” in FIG. 2. During this phase of the data filtering, a time was identified at which each septic shock subject transitioned from sepsis or severe sepsis to septic shock. This time was determined by identifying period in which a subject experienced hypotension (systolic blood pressure les then 90 mmHg for more than thirty minutes) during an interval where the subject also met the herein discussed SIRS criteria and where fluid input exceeded 600 mL during the interval one hour prior to midway through the hypotensive eposide.

Of the 17,000 subject records in the database, 459 were coded for septic shock. Of these 459 subjects, 261 met the above described data requirements. Of the 261 subjects that were included in the dataset, 250 were classified as having SIRS (either sepsis or severe sepsis), and 65 of these 250 also experienced septic shock. FIGS. 3a and 3b show data for one subject that was included in the dataset and who experienced septic shock. The vertical dashed line at the left hand side of each graph in FIGS. 3a and 3b represents the beginning of a period in which the subject is classified as experiencing SIRS. The vertical dashed line toward the right hand side of each graph represents the onset of hypotension despite fluid resuscitation, and thus septic shock.

### Example 2

### Construction of a Model to Predict When a Subject May Experience Hypotension Despite Fluid Resuscitation/Septic Shock

Six multivariate logistic regression models for predicting the onset of hypotension despite fluid resuscitation/septic shock were constructed using a machine learning approach and the dataset of Example 1.

Training datasets were created from the dataset of Example 1 and were used to construct each of the six multivariate logistic regression models through machine learning approaches. Each training set comprised a subset of the data from the dataset of Example 1, including a classification for each subject (i.e., non-shock sepsis or septic shock), a time point of interest, and a feature matrix of physiologic values associated with the time point of interest.

The time point of interest was the time preceding the onset of septic shock in the data for each subject of the training dataset, and thus the time for which the model was constructed. That is, for a 30 minute time point of interest, the model was constructed to evaluate the subject initially at a time point 30 minutes prior to the onset of septic shock. The 30 minute time of interest did not necessarily correlate to the time ahead of the onset of septic shock for which a model would predict a likelihood of a subject experiencing septic shock—rather, the time point of interest represented a starting point for analysis and building of various models in this example.

The training datasets included matrices of physiological values at the time point of interest for each subject in the dataset that experienced septic shock. Training datasets for non-shock sepsis subjects were also constructed and included matrices of physiological values taken at a random time throughout the dataset, since there was no time point of interest for these subjects. Each matrix included the three data points that preceded the time of interest for each physiological value, and percent changes between each reading. The physiological values included systolic blood pressure, pulse pressure, heart rate, temperature, respiratory rate, white blood cell count, arterial pH, pulse oximetry oxygen saturation, estimated cardiac output, and estimated total peripheral resistance.

The data was normalized to a number between 0 and 1 before being included in the matrix for each subject. FIG. 4 shows the ranges that were used for normalization. Values below or above the ranges shown in FIG. 4 were set to 0 or 1, respectively. In this regard, the normalization also acted as a filter to reduce the impact of outlying data points.

A multivariate logistic regression was then performed on the matrices to identify the k-best variables for six different time points of interest (30 minutes, 60 minutes, 90 minutes, 120 minutes, 180 minutes, and 240 minutes). In these experiments, a greedy forward machine learning method was used. During the first iteration, univariate regression models were constructed for each variable. The best classifier, as judged by the area under a receiver operating characteristic curve, was selected. In subsequent iterations, new variables were added using the same methodology. The process was stopped when improvement in the area under the receiver operating characteristic curve was less than 2%. Furthermore, variables were excluded with an exit criteria of p>0.10.

The classifiers were initially evaluated using a seven-fold cross validation method. As shown in FIG. 5, there was a downward trend in mean area under the curve as the reference time prior to the onset of septic shock increased, although the 120 minute model has an area under the curve nearly equal to that of the 30 minute model.

After the initial cross validation evaluation, models for each time point of interest were created using the whole dataset The models were then tested on the same dataset. FIG. 6 plots the receiver operating curves for each of the models, which are fairly similar. Accuracy, sensitivity, and specificity values at the threshold which maximized accuracy for this example are provided for each model in FIG. 5. The models for each time point of interest performed with accuracies in the mid to upper 80's. An overall trend of decreasing sensitivity and increasing specificity was seen in these examples as the reference time prior to onset of shock increased.

The k-best variables selected by each classification model are provided in FIG. 7, where the associated Beta coefficients, adjusted odds ratios (adj OR), and p-values are also provided. Odds ratios, as shown, were adjusted to set increment increases in the variable of interest. For example, the odds ratios given for systolic blood pressure were for 10 mmHg increases. The complete adjustment increments are provided in FIG. 8.

The model for each time point of interest identified systolic blood pressure as a good indicator for when a subject is progressing to hypotension despite fluid resuscitation/septic shock. The two models with shorter duration of time between the time point of interest and the onset of septic shock (30 and 60 minutes) show that heart rate was the second best indicator while the models with longer duration of time between the time point of interest and the onset of septic shock selected a respiratory indicator (either respiratory rate or SpO2.). Five out of the six models picked either respiratory rate or arterial pH as a preferred variable, for this example. White blood cell count entered into two of the models (60 and 180 minutes). Pulse pressure and the associated estimated cardiac output were selected for the 240 minute model.

The adjusted odds ratios aligned with clinical findings of septic shock for this example. Risk factors, as determined by the odds ratios, are provided in FIG. 8. As suggested by the adjusted odds ratios, risk for septic shock increases with a drop in systolic blood pressure, pulse pressure, SpO2, or arterial pH. Additionally, risk for septic shock increases with an increase in heart rate, respiratory rate, white blood cell count, or cardiac output. All variables selected in the models were statistically significant as indicated by the p-values, which range from less than 0.001 to 0.059.

Results from Hosmer-Lemeshow goodness-of-fit testing are provided in FIG. 9. Adequate goodness-of-fit is indicated by p-values greater than 0.05. Analysis of the calibration plots shows that two models that are not statistically good-fits may have been skewed by a few outlying points.

### Example 3

### Testing the Models Developed Under Example 2

Models for each of the time points of interest were tested in a forward, causal manner against subject records from the database that was used to construct the dataset of Example 1. These tests were performed to simulate performance in an intensive care unit setting. A schematic of the overall evaluation process is provided in FIG. 10.

The output of each model was evaluated to determine an appropriate threshold value for an output indicator—that is, when an alarm was to be issued. In some approaches, it may be desirable for an alarm to issue before an episode of hypotension despite fluid resuscitation, and before therapeutic interventions, such as the start or increase in vasopressors or inotropic agents, were administered to subjects in the database.

Five different summing algorithms were used to perform further manipulation on model output before deciding to issue a warning. These summing algorithms combined consecutive outputs from each model, which may have mitigated the effects of any single misclassification and may have captured time devolution towards hemodynamic instability. The algorithms used a range of 1 to 5 consecutive outputs from a model for input in the summing algorithm.

In these examples, an alarm was set to issue at the earliest of 18 hours prior to the onset of hypotension despite fluid resuscitation or the start or at least 100% increase in vasopressors or inotropic agents. The methodology discussed herein with respect to the septic shock onset detector, under Example 1, was used to identify hypotension despite fluid resuscitation. The start or increase in vasopressors/inotropic agents was determined using medication information that was included in each dataset, as discussed above. The occurrence of either of these events was referred to as a gold-standard episode in the example—meaning that the model predicted the onset of septic shock.

Positive and negative predictive values (PPV/NPV), which measure the proportion of patients with a positive or negative test result who are correctly diagnosed, were used to evaluate each of the models. Here, the PPV is the probability that a patient will experience a gold-standard episode given that an early warning was issued. Conversely, the NPV is the probability a patient will not experience a gold-standard episode if no warning is issued, FIG. 11 provides a graphical depiction of the definitions for true positive, false positive, true negative, and false negative occurrences. Definitions for PPV and NPV are also provided below by Eq. 7 and 8, respectively.

$$\begin{matrix}PPV = \frac{True\_Positives}{True\_Positives + False\_Positives} & Eq . 7 \\ NPV = \frac{True\_Negatives}{True\_Negatives + False\_Negatives} & Eq . 8\end{matrix}$$

Sensitivity was chosen as a measure of model performance. Sensitivity is a statistical measure of the proportion of true positives correctly identified by the classification test. In this example, the sensitivity was the proportion of episodes of hypotension despite fluid resuscitation for which the system provided prior alert, as reflected by Eq. 9.

$$Sensitivity = \frac{True\_Positives}{True\_Positives + False\_Negatives} \qquad Eq . 9$$

For this example, true positives and false negatives were defined as shown graphically in FIG. 12. These definitions were used in this calculation to prevent a skewed measure of the index (as with NPV), such that the models would not be penalized for detecting the onset of septic shock only 6 hours prior rather than the full 18 hours identified as the gold-standard for this example.

The results from Example 3 are shown in FIG. 13, which provides a breakdown of the gold-standard episodes present in the patient population. There were a total of 179 starts or 100% increases in vasopressors/inotropes within the patient population, 109 of which occurred in patients who only exhibit SIRS while 70 occurred in patients who experience hypotension despite fluid resuscitation. Additionally, 34 episodes of hypotension despite fluid resuscitation occurred amongst 26 patients.

Each model was tested with each of the summing algorithms discussed herein with respect to Eq. 2-Eq. 6. Various numbers of consecutive inputs between 2 and 5 were used for each summing algorithm. Results for the 120 minute model are discussed herein, and are represented in FIG. 14. FIG. 14 includes a plot for sample sensitivity versus PPV of the 120 minute model using Eq. 2 and a plot of sensitivity for hypotension despite fluid resuscitation (HDFR) episodes versus PPV. The models that were tested provided early warnings for hypotension despite fluid resuscitation episodes with a higher sensitivity than for the start/increase in vasopressors/inotropes. Analysis of the sensitivity versus PPV plots for various tests suggests that the 120 minute model using no summing algorithm provides beneficial results. The classification threshold with the highest clinical utility was identified as the point with a sensitivity of 82% and PPV of 67% (corresponding to a threshold of 0.87).

The models tested in this example were found to be good at identifying the occurrence of no gold-standard episodes as indicated by the high concentration of low output values in the FIG. 15(a). Conversely, some positive gold-standard data points go undetected as indicated in FIG. 15(b). However, a data point is defined, for this example, as gold-standard positive if any gold-standard episode occurs in the following 18 hours. If the episode was detected only 6 hours previous of occurrence, 12 of the remaining 18 hours of data would have been classified as false negatives. Thus, the high occurrence of low output values for positive gold-standard data points may be less important since the maximum output value in the 18 hours prior may matter more.

FIG. 16 provides the maximum classifier output value (i.e., output indicator values) in the 18 hours prior to gold-standard episodes for the 120 minute model using no summing algorithm. The top graph is a histogram of the maximum outputs 18 hours prior to the start/increase in vasopressors/inotropes. The bottom graph is for episodes of hypotension despite fluid resuscitation. These graphs suggest that this particular model was good at detecting episodes of hypotension despite fluid resuscitation.

FIG. 17 provides performance indices for models when run with an output indicator threshold of 0.87 (on a scale between 0 and 1). This threshold was found to provide good clinical utility by achieving high sensitivity while still performing with an acceptable false positive rate. The model performed at a sensitivity and specificity of 0.82 and 0.96, respectively. This resulted in a PPV and NPV of 0.67 and 0.81, respectively. The model detected 29 out of 34 episodes of hypotension despite fluid resuscitation with a mean early warning time of 582 minutes (median 600). Additionally, the model detected 124 out of 229 start/increases in vasopressors/inotropes with a mean early warning time of 507 minutes (median 528).

A sample patient run is shown in FIGS. 18a and 18b. Here, five clinical values are provided to track the state of a subject: (1) systolic blood pressure, (2) heart rate, (3) white blood cell count, (4) temperature, and (5) respiratory rate. The bottom plot shows the output of the model. Portions of the lines with dots indicate the issuance of an early warning (output>threshold). The left most vertical line denotes an episode of hypotension despite fluid resuscitation, and the next lines denote a start/increase in vasopressors/inotropes. The frequency of warnings increases in the time interval prior to the subject experiencing gold-standard episodes.

The foregoing written specification is considered to be sufficient to enable one ordinarily skilled in the art to practice the invention. The present invention is not to be limited in scope by examples provided, since the examples are intended as mere illustrations of one or more aspects of the invention. Other functionally equivalent embodiments are considered within the scope of the invention. Various modifications of the invention in addition to those shown and described herein will become apparent to those skilled in the art from the foregoing description. Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth or illustrated in the drawings. The invention is capable of other embodiments and of being practiced or of being carried out in various ways.

Also, the phraseology and terminology used herein is for the purpose of description and should not be regarded as limiting. The use of “including”, “comprising”, or “having”, “containing”, “involving”, and variations thereof herein, is meant to encompass the items listed thereafter and equivalents thereof as well as additional items.

What is claimed is:

1. A method of identifying when a subject is at an elevated risk for septic shock, the method comprising:
measuring a plurality of time varying physiological variables of the subject;
providing the plurality of time varying physiological variables to a model;
calculating, with the model, an output indicator based on the plurality of time varying physiological variables received by the model; and
identifying that the subject is at an elevated risk for septic shock when the output indicator, provided by the model, exceeds a threshold value.

2. The method of claim 1, wherein measuring the plurality of time varying physiological variables from the subject and providing the plurality of time varying physiological variables to the model comprises measuring and providing two time varying physiological variables.

3. The method of claim 2, wherein the two or more time varying physiological variables are chosen from a group comprising: systolic blood pressure, heart rate, temperature, respiratory rate, white blood cell count, arterial pH, and arterial blood oxygen saturation level.

4. The method of claim 2, wherein the two or more time varying physiological variables are chosen from a group consisting of: systolic blood pressure, heart rate, temperature, respiratory rate, white blood cell count, arterial pH, and arterial blood oxygen saturation level.

5. The method of claim 1, further comprising:
repeating the acts of providing the plurality of time varying physiological variables to the model and calculating the output indicator.

6. The method of claim 1, wherein measuring the plurality of time varying physiological variables from the subject and providing the plurality of time varying physiological variables to the model comprises measuring and providing six or more time varying physiological variables.

7. The method of claim 1, wherein calculating the output indicator comprises multiplying each of the plurality of time varying physiological variable and a corresponding constant to produce the output indicator.

8. The method of claim 1, wherein the model is a multivariate logistic regression model.

9. The method of claim 1, wherein measuring comprises measuring the plurality of time varying physiological variables about every hour.

10. The method of claim 1, wherein identifying that the subject is at the elevated risk for septic shock comprises identifying that the subject is likely to experience hypotension despite fluid resuscitation.

11. The method of claim 1, wherein the model is incorporated into a computer readable medium that, when executed by a computer, calculates the output indicator based on the plurality of time varying physiological variables received by the model.

12. The method of claim 1, further comprising:
estimating time varying physiological information of the subject based on the plurality of time varying physiological variables and providing the time varying physiological information to the model to calculate the output.

13. The method of claim 1, wherein the model has a sensitivity of greater than 0.8.

14. The method of claim 1, wherein identifying that the subject is at an elevated risk for septic shock occurs, on average, about ten hours prior to when the subject would experience septic shock.

15. A system for identifying when a subject is at an elevated risk for septic shock, the system comprising:
an input device for receiving a plurality of time varying physiological variables of the subject;
a controller that calculates, based on the plurality of time varying physiological variables and a model, an output indicator; and
an output device that provides an alert when the output indicator exceeds a threshold value indicating that the subject is at an elevated risk for septic shock.

16. The system of claim 15, wherein the plurality of time varying variables comprises two or more time varying physiological variables.

17. The system of claim 16, wherein the two or more time varying physiological variables are chosen from a group comprising: systolic blood pressure, heart rate, temperature, respiratory rate, white blood cell count, arterial pH, and arterial blood oxygen saturation level.

18. The system of claim 16, wherein the two or more time varying physiological variables are chosen from a group consisting of: systolic blood pressure, heart rate, temperature, respiratory rate, white blood cell count, arterial pH, and arterial blood oxygen saturation level.

19. The system of claim 15, wherein the plurality of time varying variables comprises four or more time varying physiological variables.

20. The system of claim 15, wherein the plurality of time varying variables comprises six or more time varying physiological variables.

21. The system of claim 15, wherein the controller is configured to compare the output indicator to a threshold value to identify whether the subject is at the elevated risk for septic shock.

22. The system of claim 15, wherein the model is a multivariate logistic regression model.

23. The system of claim 15, wherein the controller calculates the output indicator about every hour.

24. The system of claim 15, wherein the threshold value corresponds to the subject being likely to experience hypotension despite fluid resuscitation within about 10 hours, on average.

25. The system of claim 24, wherein the threshold value corresponds to the subject being likely to experience hypotension despite fluid resuscitation within about 6 hours, on average.

26. The system of claim 15, wherein the model is incorporated into a computer readable medium that, when executed by the controller, calculates the output indicator based on the plurality of time varying physiological variables and the model.

27. A method of constructing a model to identify when a subject is at an elevated risk for septic shock, the method comprising:
gathering measurements of a group of time varying physiological variables at different times from subjects that experienced at least one of SIRS, sepsis, severe sepsis, or septic shock;
identifying times at which the subjects exhibited an indicator of septic shock;
choosing combinations of the time varying physiological variables and
defining constants associated with the time varying physiological variables that when multiplied by corresponding time varying physiological variables and summed, produce an output indicator that represents when a corresponding subject was at an elevated risk for septic shock.

28. The method of claim 27, wherein defining constants associated with the time varying physiological variables includes using a multivariate logistic regression.

29. The method of claim 27, wherein the indicator of septic shock is hypotension despite fluid resuscitation.

30. The method of claim 27, wherein the indicator of septic shock is hypotension an administration of vasopressors or inotropes.

31. The method of claim 27, wherein gathering measurements of the group of time varying physiological variables includes acquiring the measurements from a database.

32. The method of claim 27, wherein the group of time varying physiological variables are chosen from a group comprising: systolic blood pressure, heart rate, temperature, respiratory rate, white blood cell count, arterial pH, and arterial blood oxygen saturation level.
